(19) United States
(12) Patent Application Publication
(10) Pub. No.: US 2001/0046526 A1
(43) Pub. Date: Nov. 29, 2001

(54) Treatment of fungal infections

(75) Inventor: Gail P. Greenfelder, Dade City, FL (US)

Correspondence Address: PEPPER HAMILTON LLP, 600 FOURTEENTH STREET, N.W., WASHINGTON, DC, 20005, US

(73) Assignee: C-Quest

(21) Appl. No.: 09/837,162

(22) Filed: Apr. 19, 2001

Related U.S. Application Data
(60) Provisional application No. 60/198,383, filed on Apr. 19, 2000.

(51) Int. Cl. A61K 35/78

(52) U.S. Cl. 424/779

(57) ABSTRACT

The composition of the invention provides formulations of extract of the Musaceas plant suitable for treatment of skin and mucosal membranes. A topical composition is disclosed for the treatment of fungal infections in a subject in need thereof in which the composition has an extract of Musaceas and a carrier. The formulation can be a liquid, a lotion, a cream, a dry powder, or a suspension, and can be used as a spray, wash, rinse, bandage ointment, or lotion. A method of treating or inhibiting fungal infections in a subject in need thereof is disclosed in which the method is administering an effective amount of a composition to the subject, where the composition has Musaceas extract and a carrier.

### 1. RELATED APPLICATIONS

This application claims priority from U.S. Provisional Application No. 60/198,383, filed Apr. 19, 2000, which is incorporated herein by reference.

### 2. FIELD OF THE INVENTION

The present invention relates generally to the prevention, inhibition and treatment of fungal infections. More specifically, this invention relates to a composition comprising Musaceas extract and its use in the prevention, inhibition and treatment of fungal infections.

### 3. BACKGROUND OF THE INVENTION

Fungal infections are ubiquitous. These infections can occur amongst the otherwise healthy, and are a particular problem for the infirm or immunocompromised. Fungal infections vary in severity from the mild discomfort associated with mucosal Candida infections to life-threatening fungal meningitis.

Humans are susceptible to a variety of fungal infections, including tinea pedis, onychomycosis, tinea cruris, tinea corpora, candidiasis, tinea versicolor, aspergilliosis, coccidiodomycosis, cryptococcal meningitis, histoplasmosis, and the like.

Tinea pedis, or athlete's foot fungus, affects over 10 percent of the United States population per year. Generally the area between the toes is affected, although the condition can spread to the sole. Athlete's foot can be passed on locker room and shower floors, as well as by the sharing of footware and socks The fungus thrives in warm, humid conditions.

Onychomycosis refers to a fungal infection under the fingernail or toenail. This condition can be particularly problematic for the diabetic individual, seriously complicating foot care.

Tinea cruris, or jock itch, also thrives in conditions of heat, moisture, and poor air circulation. Usual symptoms of this infection are intense itching and burning.

Tinea corpora, or ringworm, is caused by microscopic fungus. The infected area spreads slowly from its central starting point, and creates a slightly raised, intensely red ring surrounding a less red, flaky, itchy area. Over time the ring slowly enlarges. It can occur anywhere on the body and in multiple sites at once.

Candidiasis presents as a brownish-red, itchy discoloration affecting the underarms, comer of the mouth, rectal area, and beneath the breasts. Candida albicans also results in vaginal candidiasis or yeast infections, oropharyngeal candidiasis or thrush, and esophageal candidiasis.

Tinea versicolor actually changes the color of the skin it infects. The patches can be lighter or darker than the normal surrounding skin. This fungal infection results in mild itching and irritation.

Aspergilliosis is relatively common among neutropenic cancer chemotherapy patients and those receiving immunosuppressive therapy for organ transplants. Symptoms of this infection include cough, chest pain, difficulty breathing, fever, night sweats, sinus pain and facial swelling.

Symptoms resulting from occidiodomycosis are non-specific, and include malaise, weight loss, fatigue and cough. Infection is caused by inhalation of airborne, ineffective arthroconidia, a stage in the coccidioides immitis life cycle. The most common infection site is the lungs. In the advanced disease state infection can appear in the kidneys, spleen, lymph nodes, brain and thyroid gland.

Cryptococcal meningitis is caused by cryptococcus neoformans, a yeast like fungus found world wide, and particularly in soil contaminated with bird excrement. Symptoms of this infection include fatigue, fever, headache, nausea, seizure, and neurological changes. Where there is pulmonary infection, symptoms can include lobar or interstitial pneumonitis and pleural effusion. The infected individual can also be asymptomatic. The most common infection site is the brain, followed by the lungs.

Histoplasmosis is caused by histoplasma capsulatum a fungus endemic to the south central U.S. and South America. Patients with compromised immune systems are particularly susceptible to this infection. Symptoms of histoplasmosis include fever, weight loss, nodular or ulcerative skin lesions, respiratory complains, anemia, and enlargement of the liver, spleen and lymph nodes.

### 4. SUMMARY OF THE INVENTION

The present invention relates to compositions comprising an extract of a plant of the Musaceas family. The compositions can be, for example, in the form of powders, liquids, or ointments. The invention also relates to methods of treatment, inhibition, and prevention of fungal infections such as tinea pedis, onychomycosis, tinea cruris, tinea corpora, candidiasis, tinea versicolor, aspergilliosis, coccidiodomycosis, cryptococcal meningitis, histoplasmosis, hoof thrush, hoof rot, and the like.

One aspect of the invention provides anti-fungal compositions. Embodiments of the composition of the invention can be liquids, gels, ointments, salves, lotions, powders, dusting mixtures and bandages or compresses. In some embodiments, the anti-fungal compositions are dusting compositions comprising dried extract of Musaceas and a carrier. The dusting compositions can be for treatment of skin infections and/or skin fungus. Suitable carriers of the dusting composition can include talc, starch, modified starch, boric acid, zinc oxide, kaolin, light calcined magnesia, potassium alum, or combinations thereof.

In one aspect, the present invention provides methods for treatment of poorly healing wounds and/or for resolution of skin infections. In some embodiments, the methods comprise administering, to an affected area, an effective amount of Musaceas extract. In some embodiments, the methods further comprise administering Musaceas extract and an antibacterial agent to an affected area. In some embodiments, the methods further comprise topically administering to an affected area an effective amount of Musaceas extract together with an antibacterial agent optionally dispersed in a lipid-based carrier or other pharmaceutically acceptable carrier. The foregoing active ingredients can be administered in any sequence (e.g., sequentially) or substantially at the same time period (e.g., contemporaneously).

In one aspect, the present invention provides a treatment that can be applied as a prophylactic treatment to prevent the onset or spread of a fungal infection.

In one aspect, the present invention provides a treatment for skin wounds, whereby healing is improved. The treatment comprises application of a composition comprising an extract of Musaceas to the wound.

In one aspect, the present invention provides a method for skin softening and rejuvenation, comprising applying a composition comprising an extract of Musaceas, whereby the skin appears softer.

In one aspect, the present invention provides compositions and methods applicable to human and/or veterinary use.

Specific embodiments of the invention may be directed to one, some, or all of the above-indicated aspects, as well as other aspects ascertainable from this disclosure, and may encompass one, some, or all of the above- and below-indicated embodiments, as well as other embodiments ascertainable from this disclosure.

### 5. DETAILED DESCRIPTION OF EMBODIMENTS

### 5.1. Definitions

Lipid-Based Carrier—Any carrier, preferably a pharmacologically acceptable carrier, comprising lipid or fatty components, especially those having a hydrophobic moiety and a hydrophilic moiety. The carrier optionally enables the transdermal transport of an active ingredient from the surface of the skin to the regions of the body below the skin. Once below the skin, the active ingredient(s) may remain localized in or around the region(s) to which the combination or composition of interest, which is partially comprised of the lipid-based carrier, has been applied. Alternatively, the active ingredient(s) may become available systemically. In some embodiments, lipids include those that form membranes, bilayers, vesicles, liposomes and the like, particularly biological membranes. Examples of such membrane-forming lipids include but are not limited to phospholipids, glycolipids and cholesterol-type lipids. Specific lipids include phosphatidyl choline, phosphatidyl serine, phosphatidyl inositol, phosphatidyl inositol and the like.

Biocompatible Organic Solvent—An organic solvent, for example, comprising at least one ester of fatty acids. The fatty acids contain long chain saturated or unsaturated aliphatic groups comprising about 8 to about 50 carbon atoms. In some embodiments the long-chain groups comprise about 12 to about 30 carbon atoms. In other embodiments the long-chain groups comprise about 14 to about 24 carbon atoms. In yet other embodiments the long-chain groups comprise about 16 to about 18 carbon atoms. Examples of fatty acids include lauric, myristic, palmitic, stearic, arachidic, behenic, lignoceric, palmitoleic, oleic, linoleic, linolenic, arachidonic acid and the like. The alcohol portion of the ester group is generally a linear or branched lower alkyl group comprising about 1 to about 8 carbon atoms. Examples of the alcohol portion include methanol, ethanol, propanol, isopropanol, butanol, isobutanol, sec-butanol, tert-butanol, pentanol, isopentanol, hexanol, heptanol, octanol, isooctanol, cyclooctanol and the like. Accordingly, esters of fatty acids suitable for use as, or as part of, biocompatible organic solvents include, but are not limited to, ethyl palmitate, ethyl myristate, ethyl oleate, isopropyl palmitate, isopropyl myristate, isopropyl oleate, and the like. Other biocompatible organic solvents include, but are not limited to, isooctane and cyclooctane.

Hydroxyl Group Containing Aliphatic Organic Solvent—An organic solvent containing a hydroxyl group, including but not limited to those described in the preceding paragraph relating to the “alcohol portion of the ester group.”

Surfactant—Any compound or surface active agent that reduces surface tension when dissolved in water or water solutions, or which reduces interfacial tension between two liquids, or between a liquid and a solid. Surfactants suitable for use in the present invention include ionic and non-ionic detergents, dispersing agents, wetting agents, emulsifiers and the like. Examples of surfactants include but are not limited to the sodium salt of an N-(alkylsulfonyl)glycine (EMULSIfIER STH), the salts of linear alkyl sulfonates (LAS), the salts of alkyl benzene sulfonates (ABS), the sodium salt of dodecylsulfate (SDS), a nonionic series of 28 related difunctional block-polymers terminating in primary hydroxyl groups with molecular weights ranging from about 1,000 to over about 15,000, polyoxyalkylene derivatives of propylene glycol (PLURONIC), and the like. The surfactant used in the present invention is preferably pharmaceutically acceptable and biodegradable. In some embodiments, the surfactant is a polyol, a polyoxyalkylene, a fatty ester, a fatty acid or salts thereof. In some embodiments, the polyoxyalkylene comprises a block polymer, for example, of poly(oxyethylene-co-oxypropylene-co-oxyethylene).

### 5.2. Description of Embodiments

In a particular embodiment of the invention, the topical composition comprises Musaceas extract and, optionally, an antibacterial agent. The topical composition can further comprise an anti-inflammatory agent, for example, a non-steroidal anti-inflammatory drug (NSAID). The composition can also further comprise L-arginine, nitroglycerine, or other nitric oxide donor. In the topical composition of the present invention, the active ingredients are optionally dispersed with other active or non-active ingredients, thickeners, emollients, fillers, etc., in a carrier. Topical compositions, according to the invention, can be in different forms; for example, they can be powders, liquids, or ointments.

The carrier can be a lipid-based carrier or a non-lipid-based carrier. Non-lipid-based carriers can be any of several types known in the art, including but not limited to mucilages, gums, gels, jellies, magmas, and milks. Synthetic or partially synthetic mucilage-like substances, including polyvinyl alcohol, methyl cellulose, carboxymethylcellulose and related substances are also suitable as components of the carrier.

Lipid-based carriers can include pharmaceutically acceptable oils, waxes, fatty acids, petrolatum, membrane-forming lipids, soaps, phospholipids, and organic solvents.

The lipid-based carrier can include a membrane-forming lipid, such as phosphatidyl choline, phosphatidyl serine, phosphatidyl inositol, phosphatidyl ethanolamine, or the like. In some embodiments, the membrane-forming lipid comprises lecithin. In some embodiments of the present invention, the composition comprises Musaceas extract, a lipid-based carrier comprising at least one membrane-forming lipid, at least two biocompatible aliphatic organic solvents comprising about 8 to about 50 carbon atoms, wherein at least one of the biocompatible aliphatic organic solvents is a hydroxyl group containing aliphatic organic solvent, in which the alcohol portion comprises about 1 to about 8 carbon atoms and at least one surfactant.

According to some embodiments, the present invention relates to methods of alleviating or ameliorating the adverse or negative effects of fungal infections. Generally, the methods comprise topically administering to an affected area an effective amount of the Musaceas extract and optionally an antibacterial agent. Other methods include topical administration to an affected area of an effective amount of the Musaceas extract and antibacterial agent in a carrier and/or also, the topical administration to an affected area, of an effective amount of a non-steroidal anti-inflammatory drug (NSAID) and/or a vasodilator. A topical composition comprising the Musaceas extract and/or antibacterial agent in a lipid-based carrier is applied daily for a period of at least about a few weeks (e.g., for at least about one week, two weeks, three weeks, or four weeks) to a few months (e.g., for at least about one month, two months, or three months) to the affected area of the subject. The topical composition can be applied once daily or more than once, for example, twice, three times and up to about five times daily or as needed for the duration of the treatment period until the desired effect is obtained. The active ingredients can be topically administered sequentially, contemporaneously, or more or less contemporaneously. Contemporaneously means within about an hour.

The daily dosage of antibacterial agent ranges from about 1 to about 100 mg/kg of the subject, and can range from about 8 to about 66 mg/kg of the subject, or can range about 25 to about 50 mg/kg of the subject. The daily dosage of the NSAID ranges from about 0.6 to about 40 mg/kg of the subject, and can range from about 3 to about 26 mg/kg of the subject, or can range from about 10 to about 20 mg/kg of the subject.

Antibacterials that can be included within the composition of the present invention include, but are not limited to, the following:

Aminoglycosides such as Amikacin, Apramycin, Arbekacin, Bambermycins, Butirosin, Dibekacin, Dihydrostreptomycin, Fortimicin(s), Gentamicin, Isepamicin, Kanamycin, Micronomicin, Neomycin, Neomycin Undecylenate, Netilmicin, Paromomycin, Ribostamycin, Sisomicin, Spectinomycin, Satreptomycin, Tobramycin, Trospectomycin, and the like;

Amphenicols such as Azidamfenicol, Chloramphenicol, Florfenicol, Thiamphenicol, and the like;

Ansamycins such as Rifamide, Rifampin, Rifamycin SV, Rifapentine, Rifaximin, and the like;

β-Lactams;

Carbacephems such as Loracarbef, and the like;

Carbapenems such as Biapenem, Imipenem, Meropenem, Panipenem, and the like;

Cephalosporins such as, Cefaclor, Cefadroxil, Cefamandole, Cefatrizine, Cefazedone,

Cefazolin, Cefcapene Pivoxil, Cefclidin, Cefdinir, Cefditoren, Cefepime, Cefetamet, Cefixime, Cefmenoxime, Cefodizime, Cefonicid, Cefoperazone, Ceforanide, Cefotaxime, Cefotiam, Cefozopran, Cefpimizole, Cefpiramide, Cefpirome, Cefpodoxime Proxetil, Cefprozil, Cefroxadine, Cefsulodin, Ceftazidime, Cefteram, Ceftezole, Ceftibuten, Ceftizoxime, Ceftriaxone, Cefuroxime, Cefuzonam, Cephacetrile Sodium, Cephalexin, Cephaloridine, Cephalosporin, Cephalothin, Cephapirin Sodium, Cephradine, Pivcefalexin, and the like;

Cephamycins such as Cefbuperazone, Cefmetazole, Cefminox, Cefotetan, Cefoxitin, and the like;

Monobactams such as Aztreonam, Carumonam, Tigemonam, and the like

Oxacephems such as Flomoxef, Moxalactam, and the like;

Penicillins such as Amdinocillin, Amdinocillin Pivoxil, Amoxicillin, Ampicillin, Apalcillin, Aspoxicillin, Azidocillin, Azlocillin, Bacampicillin, Benzylpenicillinic Acid, benzylpenicillin Socium, Carbenicillin, Carindacillin, Clometocillin, Cloxacillin, Cyclacillin, Dicloxacillin, Epicillin, Fenbenicillin, Floxacillin, Hetacillin, Lenampicillin, Metampicillin, Methicillin Sodium, Mezlocillin, Nafcillin Sodium, Oxacillin, Penamecillin, Penethamate Hydriodide, Penicillin G Benethamine, Penicillin G Benzathine, Penicillin G Benzhydrylamine, Penicillin G Calcium, Penicillin G Hydrabamine, Penicillin G Potassium, Penicillin N, Penicillin O, Penicillin V, Penicillin V Benzathine, Penicillin V Hydrabamine, Penimepicycline, Phenethicillin Potassium, Piperacillin, Pivampicillin, Propicillin, Quinacillin, Sulbenicillin, Sultamicillin, Talampicillin, Temocillin, Ticarcillin, Ritipenem, and the like;

Lincosamides such as Clindamycin, Lincomycin, and the like;

Macrolides such as Azithromycin, Carbomycin, Clarithromycin, Dirithromycin, Erythromycin, Erythromycin Acistrate, Erythromycin Estolate, Erythromycin Glucoheptonate, Erythromycin Lactobionate, Erythromycin Propionate, Erythromycin Stearate, Josamycin, Leucomycins, Midecamycins, Miokamycin, Oleandomycin, Primycin, Rokitamycin, Rosaramicin, Roxithromycin, Spiramycin, Troleandomycin and the like;

Polypeptides such as Amphomycin, Bacitracin, Capremycin, Colistin, Enduracidin, Enviomycin, Fusafungine, Gramicidin S, Gramicidin(s), Mikamycin, Polymyxin, Pristinamycin, Ristocetin, Teicoplanin, Thiostrepton, Tuberactinomycin, Tyrocidine, Tyrothricin, Vancomycin, Viomycin, Virginiamycin, Zinc Bacitracin, and the like;

Tetracyclines such as Apicycline, Chlortetracycline, Clomocycline, Demeclocycline, Doxycycline, Guamecycline, Lymecycline, Meclocycline, Methacycline, Minocycline, oxytetracycline, Penimepicycline, Pipacycline, Rolitetracycline, Sancycline, Tetracycline, Cycloserine, Mupirocin, Turbin, and the like;

2,4-Diaminopyrimidines such as Brodimoprim, Tetroxoprim, Trimethoprim, and the like;

Nitrofurans such as Furaltadone, Furazolium Chloride, Nifuradene, Nifuratel, Nifurfoline, Nifurpirinol, Nifurprazine, Nifurtoinol, Nitrofurantoin, and the like;

Quinolones and Analogs such as Cinoxacin, Ciprofloxacin, Clinafloxacin, Difloxacin, Enoxacin, Fleroxacin, Flumequine, Grepafloxacin, Lomefloxacin, Miloxacin, Nadifloxacin, Nalidixic Acid, Norfloxacin, Ofloxacin, Oxolinic Acid, Pazufloxacin, Pefloxacin, Pipemidic Acid, Piromidic Acid, Rosoxacin, Rufloxacin, Sparfloxacin, Temafloxacin, Tosufloxacin, Trovafloxacin, and the like;

Sulfonamides such as Acetyl Sulfamethoxypyrazine, Benzylsulfamide, Chloramine-B, Chloramine-T, Dichloramine T, N²-Formylsulfisomidine, N⁴-β-D-Glucosylsulfanilamide, Mafenide, 4′-(Methylsulfamoyl)sulfanilanilide, Noprylsulfamide, Phthalyl-sulfacetamide, Phthalylsulfathiazole, Salazosulfadimidine, Succinylsulfathiazole, Sulfabenzamide, Sulfacetamide, Sulfachlor-pyridazine, Sulfachrysoidine, Sulfacytine, Sulfadiazine, Sulfadicramide, Sulfadimethoxine, Sulfadoxine, Sulfaethidole, Sulfaguanidine, Sulfaguanol, Sulfalene, Sulfaloxic Acid, Sulfamerazine, Sulfameter, Sulfamethazine, Sulfamethizole, Sulfamethomidine, Sulfamethoxazole, Sulfamethoxypyridazine, Sulfametrole, Sulfamidochrysoidine, Sulfamoxole, Sulfanilamide, 4-Sulfanilamidosalicyclic Acid, N⁴-Sulfanilylsulfanilamide, Sulfanilylurea, N-Sulfanilyl-3,4-xylamide, Sulfanitran, Sulfaperine, Sulfaphenazole, Sulfaproxyline, Sulfapyrazine, Sulfapyridine, Sulfasomizole, Sulfasymazine, Sulfathiazole, Sulfathiourea, Sulfatolamide, Sulfisomidine, Sulfisoxazole, and the like;

Sulfones such as Acedapsone, Acediasulfone, Acetosulfone Sodium, Dapsone, Diathymosulfone, Glucoculfone Sodium, Solasulfone, Succisulfone, Sulfanilic acid, p-Sulfanilyl-benzylamine, Sulfoxone Sodium, Thiazolsulfone, and the like;

Others such as Clofoctol, hexedine, Methenamine, Methenamine Anhydromethylene-citrate, Methenamine Hippurate, Methenamine Mandelate, Methenamine Sulfosalicylate, Nitroxoline, Taurolidine, Xibomol, and the like;

Leprostatics such as Acedapsone, Acetosulfone Sodium, Clofazimine, Dapsone, Diathymosulfone, Glucosulfone Sodium, Hydnocarpic Acid, Solasulfone, Succisulfone, Sulfoxone Sodium, and the like;

Rickettsia such as Antirickettsial, and the like;

Tuberculostatics such as p-Aminosalicyclic Acid, p-Aminosalicylic Acid Hydrazide, Benzoylpas, 5-Bromosalicylhydroxamic Acid, Capreomycin, Clofazimine, Cyacetacide, Cycloserine, Dihydrostreptomycin, Enviomycin, Ethambutol, Ethionamide, Furonazide, Glyconiazide, Isoniazid, Morphazinamide, opiniazide, Phenyl Aminosalicylate, Protionamide, Pyrazinamide, Rifabutin, Rifampin, Rifapentine, Salinazid, Streptomycin, Streptonicozid, Subathizone, Sulfoniazide, Thiacetazone, Tiocarlide, Tuberactinomycin, Tubercidin, Tuberin, Verazide, Viomycin, and the like; and

Others, including acriflavine, as well as βlactamase inhibitors, renal protectant, renal dipeptidase inhibitors, and the like.

The present invention is also directed to processes for the preparation of compositions that can be applied topically to alleviate the adverse effects of fungal infections. In particular, the combinations or compositions are obtained by preparing a Musaceas extract. Optionally, the extract is combined with an antibacterial agent. In some embodiments, the extract composition is combined with at least one NSAID and/or at least one vasodilator. The composition can also be prepared by combining an extract, and optionally an antibacterial agent, with a carrier. A second mixture is then prepared, which comprises at least one lipid and at least one biocompatible organic solvent. The first and second mixtures are then combined with mixing, preferably at or slightly below room temperature. To the resulting mixture, which contains the principal active ingredient (e.g., the Musaceas extract), an amount of a third mixture comprising water and a surfactant is then added, which amount is effective to provide a combination having a creamy texture. According to the present invention, the preferred surfactant is PLURONIC. Other methods, known in the art, of combining these ingredients to prepare topical formulations are also within the scope of the invention.

An ingredient in compositions intended for topical application can be a material that assists in causing the active ingredients to pass through the dermal layer to permit the subcutaneous attack of the condition sought to be ameliorated. Such dermal layer penetration assistants include lipids and alcohols, including water-soluble alcohols, and other mutual solvents that cause the active ingredients to have increased dermal penetrability. Examples of solvents include dimethyl sulfoxide (DMSO) and ethanol. Ethanol, for example, is readily available, inexpensive and pharmaceutically acceptable.

Another constituent of topically applicable compositions can be an emollient. Isopropyl palmitate is one example of such emollient materials. Other such materials are well known in this art and will be apparent to those of ordinary skill in the art. Surfactants are common ingredients in cremes and ointments for transdermal delivery of medicaments. Non-ionic, cationic and anionic surfactants are all considered suitable for use in the preparation of the topically applied composition of this invention. One example of a suitable surfactant is a commercially available material known by the name PLURONIC.

It is considered to be within the scope of this invention to provide additional therapeutic agents in the topically applied composition hereof in order to treat specific conditions that often accompany fungal ailments. For instance, as some of these conditions are painful, it is therefore appropriate to include topical anesthetics in the inventive compositions. Topical analgesics are suitable constituents as well. NSAIDs derived from aminoarylcarboxylic acid, arylcarboxylic acid, arylacetic acid, arylpropionic acid, arylbutyric acid, pyrazole, pyrazolone, or thiazinecarboxamides are useful additions to the creme and/or ointment spray compositions of this invention. For example, NSAIDs such as ketoprofen, ibuprofen and naproxen are used in the composition and methods of the present invention. Further, thickening agents can also be used in compositions of this invention. For example, hydroxymethyl or hydroxyethyl cellulose and the like can be used as thickening agents.

Where the topically applied composition of this invention is in the form of a foam, it is suitable to include conventional pharmaceutically acceptable blowing agents in the product. Gases such as butane, propane, pentane and mixtures thereof are known for making foams. A volatile compound, such as a light hydrocarbon or carbon dioxide, can be incorporated into the composition of this invention in a pressurized container, such as a conventional aerosol can. When the composition is released, the light hydrocarbon blowing agent expands and converts the composition of this invention into a foam for topical application.

Conventional odorants, such as perfumes, and colorants, such as food dyes, can be used in their conventional proportions for their conventional purposes. Deodorants can also be used.

In some embodiments, compositions which can offer improved transdermal transport are formed by first dissolving active components in ethanol or some other pharmaceutically acceptable solvent. Preferably this solvent is also compatible with water. This active-ingredient-containing composition is then converted into a topical creme or ointment by being admixed or combined with a lipid, surfactant, biocompatible organic solvent, water, thickeners, emollients, surfactants, and/or other conventional ingredients of a creme, ointment or foam. The resulting topical composition is then applied to an affected portion of the dermal layer.

### 6. EXAMPLES

The following are non-inclusive illustrations of the compositions and methods that are contemplated by the present invention.

### 6.1. Preparation of a Musaceas Extract Suitable for Oral and Topical Application

The trunks of Musaceas plants, including but not limited to bananas (Musa cavendish enano) and plantain (Musa paradisiaca), are crushed and extracted in a mill. In one embodiment the bark can first be removed from the trunk and the debarked trunk used for milling and extraction. Musaceas here refers to the taxonomic family Musaceae which has three genera: Musa, Musella and Ensete. The three genera have about 42 species. The species include: Musa ensete, Ensete maurelii, Musa acuminata, Musa acuminata x balbisiana, Musa airori, Musa bakeri, Musa balbisiana, Musa banksii, Musa basjoo, Musa beccarii, Musa charlioi, Musa coccinea, Musa errans, Musa fehi, Musa fitzalanii, Musa textilis, Musa glauca, Musa halabanensis, Musa hillii, Musa jackeyi, Musa liukiuensis, Musa maculata, Musa mannii, Musa martinii, Musa minor, Musa nana, Musa nepalensis, Musa ornata, Musa paradisiaca, Musa rosacea, Musa salaccensis, Musa sinensis, Musa troglodytarum, Musa velutina, Musa wilsonii, Musa zebrina, Musella lasiocarpa, and Ensete ventricosum.

Those skilled in the art will recognize that the above species are members of the superorder Zingberiflorae. Similarly, those skilled in the art will recognize that common names of the Zingberiflorae and varietal names are related to the plants described above by genus and species.

Any of a number of commercial or custom milling or grinding devices can be used, including a trapegee or sugar cane mill or grinding machine. The extract or juice of the trunks is collected and, optionally, filtered. The filtration can be through successive layers of cheesecloth, muslin, paper, or combinations thereof. Additional methods of extraction and filtration are known to those skilled in the art and are also suitable.

The extract can be used fresh, stored by refrigeration, or preserved for further processing. The extract can also be pasteurized, or, alternatively, sterilized by, for example, filtration through membranes that exclude bacteria. One suitable method of pasteurization includes heating the extract at about 60° C. for about a half-hour. Further processing can include drying as disclosed in Example 6.3, concentrating, or, alternatively, combining the extract , the concentrate, or the dry powder with other desirable components, including, optionally, ingredients discussed above such as a sweetener, an oil, an emulsifier, an emollient, a wetting agent, a detergent, a penetrant, ethanol, colorants, flavors, fragrances, preservatives, and/or carrier powder. The extract can also be an effective mixture diluted with water.

Based on the description herein, a person of ordinary skill will understand that compositions within the scope of the invention include any composition with the Musaceas extract in an amount effective for the conditions disclosed herein. Thus the composition may include additional ingredients known in the art suitable for various implementations. For example, can include ingredients used to formulate lotions, ointments, or any formulation disclosed herein. Examples of compositions include those formed by the ingredients below in any combination in the following ranges:

(a) about 40% to about 100% by weight of Musaceas extract, and optionally, concentrated extract;

(b) about 0% to about 40% by weight of a sweetener;

(c) about 0% to about 20% by weight of an oil;

(d) about 0% to about 15% by weight of an emollient, emulsifier, a surfactant, a wetting agent, a detergent, a penetrant, or combinations thereof;

(e) about 0% to about 60% by weight of non-extract water;

(f) about 0% to about 60% by weight of ethanol;

(g) about 0% to 5% by weight of flavors or fragrances;

(h) about 0% to about 10% by weight preservatives; and

(i) about 0% to about 80% by weight carrier powder.

The sweetner can be calcium saccharin, Aspartame®, sugar, maltodextrin, polycose (a synthetic polyglucose), dextrose, sucrose, maltose, galactose, fructose, corn syrup, corn syrup solids, molasses, honey, mannose, lactose, dextrin, high fructose corn syrup, invert sugar, honey, molasses, lactose, or combinations thereof.

The oil can be any suitable oil, including jojoba oil, various hydrocarbon oils, higher fatty acids, higher alcohols, esters, fat/oils, waxes, silicone and fluorine oil such as squalane, liquid paraffin, vaseline, microcrystalline wax, ozokerite, ceresin, myristic acid, palmitic acid, stearic acid, oleic acid, isostearic acid, behenic acid, cetyl alcohol, stearyl alcohol, oleyl alcohol, batyl alcohol, cetyl-2-ethylhexanoate, 2-ethylhexyl palmitate, 2-octyldedecyl myristate, neopentyl glycol-2-ethylhexanoate, glyceryl trioctancate, 2-octyldodecyl oleate, isopropyl myristate, myristyl myristate, glyceryl triisostearate, glyceryl trioleate, coconut oil fatty acid triglyceride, olive oil, avocado oil, sunflower oil, safflower oil, tsubaki oil, shea butter, macadamia nut oil, mink oil, lanoline, castor oil, Japanese core wax, dimethyl polysiloxane, ring dimethyl polysiloxane, methylphenyl polysiloxane, silicone resin, polymer silicone, polyether modified silicone, amino denatured silicone, perfluoro polyether and perfluorocarbon, humectants such as ethylene glycol, diethylene glycol, 1,3-butylene glycol, glycerine, hexamethylene glycol, isoprene glycol, polyethylene glycol, propylene glycol, dipropylene glycol, and diglycerine.

The emollient, emulsifier, surfactant, wetting agent, detergent, or penetrant, can be glycerine isopropyl palmitate, non-ionic surfactant, cationic surfactant, anionic surfactant, PLURONIC, dimethyl sulfoxide, and others as known to a person of ordinary skill in the relevant art.

The liquid carrier can be water, alcohol, or any pharmaceutically acceptable fluid.

The flavors or fragrances can include any known in the art, including, but not limited to, mint oil, fruit flavors, nut flavors, bean flavors (including coffee, vanilla, and chocolate), vegetable flavors, herb flavors (including tea), flower flavors (including rose and nasturtium), butter and cream flavors, caramel flavor, spice flavors, savory flavors, and meat flavors. For example, the flavor can be lemon, orange, grapefruit, apple, pear, grape, raspberry, strawberry, blackberry, boysenberry, black raspberry, blueberry, cherry, sour cherry, lime, banana, kiwi, mango, papaya, passion fruit, guava, citron, lemon zest, orange zest, almond, pecan, walnut, black walnut, hazelnut or filbert, cashew, macadamia, Brazil nut, pistachio, milk chocolate, bittersweet chocolate, malted chocolate, coffee, spearmint, peppermint, wintergreen, black tea, oolong tea, green tea, herb tea (including, but not limited to hibiscus tea, chamomile tea, and rose hips tea). The flavors or fragrances can be natural, artificial, or both.

The preservatives can be any known in the art, including, but not limited to, methylparaben, prolylparaben, potassium sorbate, sodium benzoate, and butylated hydroxytoluene.

The dry carriers can include, but are not limited to, talc, starch, modified starch, boric acid, zinc oxide, kaolin, light calcined magnesia, potassium alum, or combinations thereof.

In one embodiment (Formula A), suitable for topical application to the skin, the composition comprises:

(a) about 95% by weight of Musaceas extract or Musaceas concentrate;

(b) about 4% by weight of a skin penetrant, or absorption enhancer;

(c) about 0.5% by weight colorant; and

(d) about 0.5% by weight preservatives.

In another embodiment (Formula B), suitable for use as an oral antifungal gargle or mouth wash, the composition comprises:

(a) about 97% by weight of Musaceas extract or Musaceas concentrate;

(b) about 2% by weight of a sweetener or sweetener solution;

(c) about 0.8% by weight of flavors and fragrances; and

(d) about 0.2% by weight preservatives.

In yet another embodiment (Formula C), suitable for use as an anti-yeast vaginal douche, the composition comprises:

(a) about 92% by weight of Musaceas extract;

(b) about 2% by weight of a wetting agent;

(c) about 5% by weight of fragrances or solution of fragrances; and

(d) about 1% by weight preservatives.

In still another embodiment (Formula D), suitable for oral or intragastric use for fungal infections, the composition comprises:

(a) about 70% by weight of Musaceas extract

(b) about 21% by weight of a sweetener or sweetener solution, e.g., sorbitol;

(c) about 8% by weight of an oil, for example, castor oil;

(d) about 0.2% by weight preservatives, for example, methylparaben or butylated hydroxytoluene.

In yet another embodiment (Formula E), suitable for either topical or oral use, the composition comprises:

(a) about 99% by weight of Musaceas extract;

(b) about 0.01% by weight of saccharine;

(c) about 0.01% by weight cherry flavor;

(d) about 0.1% by weight glycerin;

(e) about 0.2% by weight methylparaben, propylparaben, and potassium sorbate; and

(f) about 0.3% by weight water.

Other formulations will be evident to those skilled in the formulary's art.

### 6.2. Preparation of Soothing Skin Creme with Musaceas Extract

A soothing creme formulation (Formula F) of the instant invention can be used for athlete's foot and other skin fungal infections. The creme is formulated with Musaceas extract, concentrated Musaceas extract, or Musaceas powder. Additional components can be added to achieve a product suitable for topical skin application to dry skin, cracked skin, skin with rash, infected skin, and skin with combinations of problems. An embodiment comprises:

(a) about 73% by weight of Musaceas extract;

(b) about 4% by weight of an emollient, e.g. glycerol;

(c) about 6% by weight of oil, e.g. jojoba;

(d) about 10% by weight dimethylpolysiloxane

(e) about 5.8% by weight of an emulsifier, e.g. Tween 20;

(f) about 1 % by weight of a fragrance, e.g. banana oil; and

(g) about 0.2% by weight of preservatives, e.g. methylparaben and propylparaben.

The materials (a), (b), (e), (f) and (g) can be mixed, heated and dissolved, and the temperature can be kept at about 70° C. to obtain the water phase. The water phase can be added to the oil and dimethylpolysiloxane phase and the mixture can be thoroughly emulsified with an emulsifier. After the emulsification, the emulsion can be cooled while being stirred. When the temperature falls to lower than about 35° C., the mixture can be poured into a container and allowed to cool and harden to obtain the cream. As a substitute for the aforementioned jojoba oil, other ingredients which are typically blended into a cosmetic creme can be blended into the medicinal creme of the present invention. In addition to the specific ingredients listed in the above formulation, pigments can be added, including talc, muscovite, synthetic mica, phlogopite, lepidolite, black mica, lithia mica, sericite, kaolin, titanium dioxide, titanium oxide-coated mica, titanium oxide-coated talc, titanium oxide-coated oxybismuth chloride, fish scale flakes and colored titanium oxide-coated mica, metal powder pigments such as aluminum powder and copper powder, inorganic pigments such as red iron oxide, yellow iron oxide, black iron oxide, carbon black, chrome oxide, chrome hydroxide, cobalt titanate, ultramarine blue, Berlin blue, zinc white, bentonite, barium sulfate, metal soap, silious earth, aluminum silicate, strontium silicate, metal salts of tungstic acid, calcium carbonate, magnesium carbonate, chrome oxide, chrome hydroxide, alumina, silica, hydroxyapatite, boron nitride and zeolite, organic powder such as nylon powder, PMMA powder, polystyrene powder, polyethylene powder, teflon powder, polyester powder and cellulose powder, organic pigments such as red 201, red 202, red 204, red 205, red 220, red 226, red 228, red 405, orange 203, orange 204, yellow 205, yellow 401 and blue 404, powder of organic pigments such as zirconium, barium or aluminum lakes of red 3, red 104, red 106, red 227, red 230, red 401, red 505, orange 205, yellow 4, yellow 5, yellow 202, yellow 203, green 3 and blue 1 natural colors such as chlorophyll and β-carotene.

### 6.3. Preparation of Dry Powder Formulation of Musaceas Extract for Use as a Dusting Powder or Ingredient

Extract of Musaceas can be dried by one of several methods. For example:

Method One: The extract is warmed by electric coils or fossil fuel burners while dry air is passed over the surface of the extract and, optionally, bubbled through the extract. The temperature in the extract is maintained between about 25° C. and about 100° C., for example between 55° C. and 70° C., during the heating stage of the drying process. When the extract is suitable concentrated, final drying can be achieved by a repeated cycle of Method One, by Method Two, or by Method Three.

Method Two: The extract or a concentrated extract is aerosolized by passing the extract through a plurality of small orifices under high pressure to produce a fine spray or mist. The mist is introduced into a chamber maintained under reduced pressure to partially or completely remove the water in the extract. The process is repeated, as necessary, until a dry powder or concentrated solution is obtained. The concentrated solution is either again subjected to the spray drying procedure or freeze-dried as in Method Three.

Method Three: The extract or a suitably concentrated extract is freeze-dried. Layers about 1 cm thick are frozen at −20° C. by exposing the extract in shallow trays to commercial freezer temperatures. The frozen extract layer is then roughly broken into coarse chips and the chips exposed to a vacuum in a commercial apparatus to remove the water. The result is a friable dry powder that is readily mixed with a suitable dry carrier, for example powdered talcum, to produce a formulation suitable for dusting feet, fur, bedding and other applications.

The dried extract can be reconstituted to form a “concentrate.” The concentrate can be about two, about three, or about four times the concentration of the original extract. The concentration can be standardized by weight of solids, by optical absorbance, by assay of components, or by any other method known in the art. A “concentrate” can also be prepared by incomplete drying of the extract.

### 6.4. Preparation of a Dry Powder Formulation of Musaceas Extract

A dry powder formulation of the instant invention can be used for athlete's foot and other skin fungal infections. The powder is formulated with dry Musaceas powder. Additional components can be used to achieve a product suitable for topical skin application to dry skin, cracked skin, skin with rash, infected skin, and skin with combinations of problems. An embodiment (Formula G) comprises, by weight:

(a) about 50% dried Musaceas extract

(b) about 2% zinc oxide

(c) about 20% kaolin

(d) about 2% light calcined magnesia

(e) about 20% talc

(f) about 2% potassium alum

(g) about 2% magnesium stearate

(h) about 0.05% methyl para-hydroxybenzoate

(i) about 0.05% propyl para-hydroxybenzoate

(j) about 0.03% butyl para-hydroxybenzoate, and

(k) about 2% benzoic acid.

The dry materials are mixed by methods well known to those in the art, for example, in a drum mixer.

### 6.5. Treatment of Athlete's Foot Fungus

A 50-year-old pharmacist drank 1.3 ml per kg of body weight of a liquid embodiment of the invention comprising extract of Musaceas according to Formula D in connection with the treatment of a different disease. He was surprised and delighted to observe that his long-standing affliction with athlete's foot (tinea pedis) was completely resolved. He continued the oral treatment for six months with no return of the fungus.

### 6.6. Treatment of Athlete's Foot Fungus in Another Individual

An individual with a history of persistent athlete's foot fungus (tinea pedis) resistant to conventional treatment with foot powder can apply the powdered embodiment of the invention, Formula G, between her toes and in her shoes from about once to about four times daily for about five weeks, then switch to a maintenance schedule of once per day.

### 6.7. Treatment of Scalp Ringworm

Individuals presenting symptoms of ringworm can be enrolled in a serial, dermatologist-directed study. Half of the affected area can be treated with the creme of the invention, Formula F, for two weeks and compared to the untreated area. A thin film of the creme can applied to the affected area such that about 0.5 g of creme is applied per day per patient. After two weeks, in response to any beneficial results in the treated area, treatment of the entire affected area can be initiated until the infection resolves, and for one week thereafter.

### 6.8. Treatment of Open and Weeping Fungal Infection Sores

A 12-year-old sports enthusiast returned from summer camp with a weeping sore on his leg that appeared to be associated with a fungal infection. The sore was resistant to conventional treatment and was treated twice daily for two weeks, until the infection resolved, with a liquid embodiment of the invention comprising Formula E: 99% Musaceas extract, and the remainder diluent and sweetener.

### 6.9. Treatment of Hoof Rot in a Pony

A Shetland pony with tender feet and presenting with a frog with exudate and pungent odor can be treated as follows. Initially, the hooves and frog can be thoroughly cleaned with a hoof pick and clean water. Then a modified creme formulation, Formula F, comprising 74% Musaceas extract can be applied liberally, that is, at about two grams per foot. The creme can be reapplied daily for four weeks. Moreover, the stable bedding can be changed to fresh, dry bedding dusted with the dry powder of the invention, where one-half pound of 60% powder is mixed with 100 pounds of wheat straw. Sawdust impregnated with Musaceas extract at a ratio of one gallon to 100 pounds, and then dried, can also be used. The pony's feet are to be kept dry. On occasion, after exercising in muddy fields, the pony's hooves and frog can be cleaned and the creme of the invention is applied.

### 6.10. Treatment of Fingernail Fungus

An individual with long-standing fingernail fungus can be treated with a variant creme of the invention comprising 65% (w/w) Musaceas extract, 4% (w/w) dried Musaceas extract powder, 4% (w/w) glycerol, 10% (w/w) jojoba oil, 10% (w/w) dimethylpolysiloxane, 6% (w/w) Tween 20, 0.7% (w/w) almond oil fragrance, 0.1% propylparaben, and 0.2% methylparaben. The creme can be applied four times daily by rubbing into the skin adjacent to the nail. The treatment can be continued for 12 weeks or until the symptoms are resolved.

### 6.11. A Spray Formulation with Musaceas Extract and Antibiotic

The composition of the invention can also be formulated for spray application. Application as a spray has home and veterinary advantages in ease and rapidity of use. Several formulations are provided below:

| | Amounts in Grams | | | | |
|---|---|---|---|---|---|
| | Example No. | | | | |
| Ingredients | 1 | 2 | 3 | 4 | 5 |
| dry Musaceas extract | 2.5 | 2.5 | 5 | 2.5 | 5 |
| Cephalexin | 0 | 30 | 0 | 30 | 30 |
| ethanol (ml) | 5 | 30 | 30 | 30 | 30 |
| lecithin | 0 | 30 | 30 | 30 | 30 |
| Pluronic | 0 | 10 | 10 | 15 | 20 |
| isopropyl palmitate | 0 | 30 | 30 | 30 | 30 |
| Ketoprofen | 12 | 12 | 12 | 12 | 12 |
| DMSO (ml) | 0 | 0 | 0 | 10 | 0 |
| Nitroglycerine | 0 | 0 | 1.2 | 2 | 0 |
| water, q.s. (total g) | 120 | 120 | 120 | 120 | 120 |

The spray formulation can be applied with a hand pump, pressurized aerosol can, electric pump, or any method known in the art. Typically, sufficient spray is applied to visibly wet the skin or fur. Variations in the formula will be apparent to one skilled in the art of formulary.

### 6.12. Impregnated Bandages of the Invention

Bandages can be impregnated with the formulation of the invention for topical, extended duration application. Suitable formulations include formulas 2 and 3 of Example 5.11 and the creme of Example 5.2 although the powder of Example 5.4 can also be equally suitable. The pad of the bandages can be made of vegetable or synthetic fiber, including but not limited to cotton and teflon. Optionally, an adhesive means such as adhesive tape can be attached to the fiber pad to maintain the position of the pad against the skin and/or wound.

### 6.13. Use for Skin Rejuvenation

An individual can apply the compositions of the invention topically to the skin to rejuvenate the skin, soften rough skin and produce a younger dermal appearance. In particular, Formula E is applied as a wash or left in contact with the skin for several minutes once, twice, or more often each day. Compresses of paper or cloth, e.g., a washcloth, can be soaked in Formula E and applied to the skin. As a consequence the skin becomes softer.

It should be apparent to one of ordinary skill in the art that other embodiments can be readily contemplated in view of the teachings of the present specification. Such other embodiments, while not specifically disclosed nonetheless fall within the scope and spirit of the present invention. Thus, the present invention should not be construed as being limited to the specific embodiments described above.

It is claimed:

1. A composition comprising:
an extract of a plant of the Musaceas family in an amount effective for treatment, inhibition or prevention of fungal infections.

2. A composition according to claim 1 further comprising at least one agent, in which the agent is chosen from anti-inflammatory, skin permeant, vasodilator, and antibiotic agents.

3. The composition of claim 1 in which the plant is chosen from Musa paradisiaca (plantain) and Musa cavendish enano (banana).

4. A composition according to claim 3, comprising the extract from Musa paradisiaca and an extract from Musa cavendish enano.

5. The composition of claim 1 in which the extract is a filtered extract.

6. The composition of claim 2 in which the skin permeant is dimethyl sulfoxide.

7. The composition of claim 1 in which the carrier comprises a surfactant.

8. An anti-fungal dusting composition comprising:
dried extract of Musaceas and a carrier.

9. The composition of claim 8 in which the carrier is talc, starch, modified starch, boric acid, zinc oxide, kaolin, alum, magnesia, or combinations thereof.

10. A topical formulation comprising:
about 40% to about 100% by weight of Musaceas extract, Musaceas concentrate, or Musaceas powder;
about 0% to about 15% by weight of an emulsifier, an emollient, a wetting agent, a detergent, a penetrant, or combinations thereof;
about 0% to about 80% by weight of non-extract liquid carrier;
about 0% to 5% by weight of colorants, flavors or fragrances; and
about 0% to about 5% by weight preservatives, suitable for topical application.

11. The formulation of claim 10 in which the formulation is adapted for application to hooves, nails, skin, or combinations thereof.

12. A therapeutic bandage comprising a pad and an effective amount of an extract of a plant of the Musaceas family.

13. The bandage of claim 12 in which the pad is a cotton or synthetic fiber pad.

14. A method of treating, inhibiting, or preventing a fungal infection in a subject in need thereof comprising:
administering to the subject an effective amount of a composition comprising an extract of a plant from the family Musaceas, whereby the fungal infection is inhibited or prevented.

15. The method according to claim 14 in which said administration is oral, vaginal or topical.

16. The method according to claim 14 in which the fungal infection afflicts dermal tissue, subdermal tissue, mucosal membranes, or combinations thereof.

17. The method according to claim 14 in which the fungal infection is tinea pedis, onychomycosis, tinea cruris, tinea corpora, candidiasis, tinea versicolor, aspergilliosis, coccidiodomycosis, cryptococcal meningitis, histoplasmosis, hoof thrush, hoof rot, or combinations thereof.

18. The method according to claim 14 in which said composition comprises:
(a) about 40% to about 100% by weight of Musaceas extract;
(b) about 0% to about 40% by weight of a sweetener;
(c) about 0% to about 20% by weight of an oil;
(d) about 0% to about 15% by weight of an emulsifier, an emollient, a wetting agent, a detergent, a penetrant, or combinations thereof;
(e) about 0% to about 60% by weight of non-extract water;
(f) about 0 to about 60% by weight of ethanol;
(g) about 0 to 5% by weight of flavors or fragrances;
(h) about 0 to about 10% by weight preservatives; and
(i) about 0 to about 80% carrier.

19. The method of claim 18 in which the carrier is a solid or a liquid.

20. The method of claim 19 in which the solid is in powder form.

21. A method of treating the hooves of an animal suffering from a fungal infection comprising administering to the feet or hooves of the animal a composition comprising fiber comprising extract of a plant of the Musaceas family.

22. The method of claim 21 in which the fiber comprises sawdust, straw, hay, or wood chips.

23. A method of preparing a composition for treating a fungal infection comprising combining an extract of Musaceas with non-extract water, ethanol, a flavor, a fragrance, a preservative, an antibiotic agent, an anti-inflammatory agent, a skin permeant, a vasodilator, a sweetner, an oil, an emulsifier, an emollient, a wetting agent, a detergent, a penetrant, a carrier, or combinations thereof.

24. A method of softening skin comprising topical application of a composition comprising an extract of Musaceas, whereby the skin appears softer.

25. A method of treating skin wounds comprising application of a composition comprising an extract of Musaceas, whereby healing is improved.

26. A composition comprising:
a pasteurized extract of the trunk of a plant of the Musaceas family.

27. A method of treating, inhibiting, or preventing a fungal infection in a subject in need thereof comprising:
administering to the subject an effective amount of a composition comprising an extract of a plant from the family Musaceas, whereby the fungal infection is inhibited or prevented

28. A composition suitable for use as an oral antifungal gargle or mouth wash comprising:
(a) about 97% by weight of Musaceas extract or Musaceas concentrate;
(b) about 2% by weight of a sweetener or sweetener solution;
(c) about 0.8% by weight of flavors and fragrances; and
(d) about 0.2% by weight preservatives.

29. A composition suitable for use as an anti-yeast vaginal douche as an antifungal comprising:
(a) about 92% by weight of Musaceas extract;
(b) about 2% by weight of a wetting agent;
(c) about 5% by weight of fragrances or solution of fragrances; and
(d) about 1% by weight preservatives.

30. A composition suitable for either topical or oral use as an antifungal comprising:
(a) about 99% by weight of Musaceas extract;
(b) about 0.01% by weight of saccharine;
(c) about 0.01% by weight cherry flavor;
(d) about 0.1% by weight glycerin;
(e) about 0.2% by weight methylparaben, propylparaben, and potassium sorbate; and
(f) about 0.3% by weight water.

31. A composition suitable for topical use as an antifungal lotion or creme comprising:
(a) about 73% by weight of Musaceas extract;
(b) about 4% by weight of an emollient;
(c) about 6% by weight of oil;
(d) about 10% by weight dimethylpolysiloxane
(e) about 5.8% by weight of an emulsifier;
(f) about 1% by weight of a fragrance; and
(g) about 0.2% by weight of preservatives.

32. A composition suitable for topical use as an antifungal powder comprising, by weight:
(a) about 50% dried Musaceas extract
(b) about 2% zinc oxide
(c) about 20% kaolin
(d) about 2% light calcined magnesia
(e) about 20% talc
(f) about 2% potassium alum
(g) about 2% magnesium stearate
(h) about 0.05% methyl para-hydroxybenzoate
(i) about 0.05% propyl para-hydroxybenzoate
(j) about 0.03% butyl para-hydroxybenzoate, and
(k) about 2% benzoic acid.
